(19) United States
(12) Patent Application Publication
(10) Pub. No.: US 2024/0207640 A1
(43) Pub. Date: Jun. 27, 2024

(54) SURFACE BRACHYTHERAPY DEVICE, APPLICATOR, AND METHODS THEREFOR

(71) Applicants: COLORADO SCHOOL OF MINES, Golden, CO (US); THE REGENTS OF THE UNIVERSITY OF COLORADO, A BODY CORPORATE, Denver, CO (US)

(72) Inventors: Frédéric Sarazin, Lakewood, CO (US); Jeramy Zimmerman, Golden, CO (US); Quentin Diot, Aurora, CO (US); David Westerly, Aurora, CO (US)

(21) Appl. No.: 18/396,386

(22) Filed: Dec. 26, 2023

Related U.S. Application Data
(60) Provisional application No. 63/477,321, filed on Dec. 27, 2022.

(51) Int. Cl. A61N 5/10

(52) U.S. Cl. CPC A61N 5/1028; A61N 2005/1094

(57) ABSTRACT

A device for surface brachytherapy includes a base configured for positioning a source with respect to a target area, an aperture defined in the base, and a shield body configured for coupling with the base, so that the source is secured within the base proximate or adjacent the aperture. The aperture is configured to transmit radiation emitted by the source, where the radiation is directed toward the target area. The base and shield body are configured to absorb at least a portion of the radiation emitted by the source, where the radiation is not directed toward the target area.

### CROSS-REFERENCE TO RELATED APPLICATION

This application claims priority to U.S. Provisional Application Ser. No. 63/477,321, filed Dec. 27, 2022, “Surface brachytherapy Device, Applicator, and Methods therefor,” which is incorporated by reference herein, in the entirety and for all purposes.

### FIELD

The technology described here relates generally to radiation therapy, including, but not limited to, surface brachytherapy techniques for treating skin cancer with selected radioisotopes. More generally, this technology relates to systems, methods and devices for radiation treatment.

### BACKGROUND

Skin cancers affect millions of Americans each year, and many times that number worldwide. Considering both melanoma and non-melanoma forms, skin cancer is generally considered the most common form of the disease. Millions of cases are treated each year in the U.S alone, at a cost of billions of dollars annually. The occurrence rate also scales with age, imposing an increased burden on already over-taxed medical infrastructures, and related social service programs.

Age and cumulative sun exposure are primary drivers of non-melanoma skin cancers (NMSC), generally dominated by basal cell carcinoma (BCC) and squamous cell carcinoma (SCC). While these are typically considered manageable conditions they may still involve complications, including not only skin lesions and other evident cosmetic effects, but also discomfort, pain, infection, scarring and potential disfiguration, if not treated appropriately. If untreated, they can develop into much more serious and potentially fatal conditions.

Decreasing unprotected sun exposure can reduce the risk of developing skin cancer, but outdoor activities also have substantial benefits for general health and well-being, and from a practical sense the exposure will never go to zero. As the population ages, therefore, the incidence rate for skin cancers will likely remain a significant health care concern.

Current treatment options for skin cancer include surgical, cryogenic, chemical, and radiological methods (radiotherapy), including external beam radiation and surface brachytherapy. Common surgical forms include excision of the evident lesions, and physical examination (biopsy) to confirm complete removal, and check for the presence of cancerous cells. Less invasive methods applicable to early-stage, lower-risk conditions include curettage and cautery (cauterization), a form of electrosurgery adapted for the removal of benign surface lesions.

Microsurgery (e.g., Mohs' micrographic surgery or MMS) can provide cosmetic benefits, with a comparable or even reduced recurrence rate, but this technique can also be substantially more time consuming (and costly), and may not be suitable for all patients, depending on overall health. The more common excision procedures, on the other hand, may also require skin grafting, and may not be well suited for sensitive skin areas including the nose, lips, areas around the eyes, and other regions of the face, which are more prone to exposure.

Topical chemotherapy and immunotherapy techniques typically confine treatment to the area of known lesions, reducing the risk of side effects (e.g., as compared to injection or oral delivery). Topicals may also be more beneficial for lower-risk (e.g., pre-cancerous) conditions. In photodynamic therapy (PDT), a photoreactive agent is applied directly onto (or injected into) the site of the lesion, and then exposed to selected light frequencies. In response, the agent generates reactive oxygen species (ROS), unstable molecules that can react with adjacent cellular components, and destroy cancerous cells. These methods also come with side effects, however, including skin irritation, erythema, itching, and pain and sun sensitivity at the treatment site. In addition, these methods are generally effective only for primarily superficial cancers that do not extend beyond the epidermis.

Radiotherapy treatments can avoid some of these issues, as well as the need for surgery, and may also be beneficial for patients with other health conditions (e.g., the use of blood thinners, infection, immunodeficiency, or other contraindications), and in the case of larger lesions, the surgery itself can become substantially more invasive and complex. Radiological treatments include both external beam radiation and surface brachytherapy; e.g., using x ray (or x-ray), gamma ray (or gamma-ray), and low-energy electron sources disposed on the skin surface, adjacent to the lesion, using a source adapted to treat the particular corresponding tumor cells.

Because x rays, gamma rays, and high-energy electrons are penetrating radiation, radiation therapy techniques require careful dose analysis and shielding in order to reduce the risk to adjacent healthy tissues. The process is commonly divided over several sessions, increasing overall treatment time, but decreasing short-term exposure risks. Traditional brachytherapy techniques can also have side effects, including erythema (redness, discoloration) and desquamation (peeling, and loss of upper skin layers), and the recurrence rate can be comparable to that of traditional and micrographic surgical techniques.

For all of these reasons, there is an ongoing need for improved skin cancer prevention, including better public understanding of the causes of skin cancer, and the easiest ways to prevent it. There is also a need for improved therapeutic techniques, in order to address the substantial number of cancers that will still inevitably occur, even when protective measures are taken. More specifically, there is a need for more effective and efficacious surface brachytherapy techniques, which can minimize exposure levels and reduce undesirable side effects, and which are adaptable to both early and later-stage conditions, including lesions of varying size, shape, depth, and type, in a wider range of patient populations.

The information included in this background, including any references cited and any description or discussion thereof, is included for technical reference purposes only. The background information and subject matter is not intended to and should not be regarded as limiting the scope of the invention, which is defined by the language of the claims.

### SUMMARY

System and methods for surface brachytherapy are described. For example, a brachytherapy device can include a base configured for positioning a radiation source with respect to a target area, an aperture defined in the base, and a shield body configured for coupling with the base, so that the source is secured within the base proximate or adjacent the aperture. The aperture is configured to transmit radiation emitted by the source, where the radiation is directed toward the target area. The base and shield body are configured to absorb at least a portion of the radiation emitted by the source, where the radiation is not directed toward the target area.

The base can include an adapter or interface portion with the aperture defined therein, and a shield guide portion coupled to or defined on the adapter or interface portion, opposite the aperture. The aperture, adapter or interface portion is used for positioning the source with respect to the target area, and the shield guide portion is adapted for coupling the shield body with the base.

A collimator can be disposed between the source and the patient in order to define the aperture, or the physical geometry and extent of the source or source assembly (or both) may define the effective geometry of the aperture. Shielding can be disposed on the upper surface of the source, opposite the aperture, about a perimeter of the source, and/or in the shield body. The collimator and shielding can be formed of a material with higher density or greater average atomic number Z than that of the base and shield body, in order to preferentially absorb penetrating radiation, including but not limited to high-energy beta particles, high-energy x rays and gamma rays.

Magnets, flexures, tabs, prongs or similar resiliently biased engagement structures can be used to releasably couple the shield body with the base. The engagement features can be configured to generate an audio, visual or haptic signal, indicating that the shield body has been properly attached and the source is secured within the base.

An attachment system can be coupled to the base to releasably secure the device to one or more anatomical features adjacent the target area, for example using a flexible fabric, a flexible band, a flexible tape, a woven or non-woven fabric, or other suitable attachment. Mechanical attachment features may also be used, or magnetic coupling elements. The bottom surface of the base can be contoured according to a particular anatomical geometry, for example to position the device adjacent facial features like the lips, eyes, or nose, or adjacent the ear, head, neck, chest, torso, pelvic region, limb, hand or foot, digit, or other body structure. The outer footprint of the base can also vary according to the anatomical geometry.

Methods for using the device include positioning the base with respect to a target area on a subject, securing the device to one or more anatomical features, and inserting the source into the base, where radiation is directed from the source toward the target area through the aperture. The shield body can then be coupled to the base, absorbing radiation that is not directed to the target area.

### BRIEF DESCRIPTION OF THE DRAWINGS

FIG. 1A is a top plan view of a surface brachytherapy device.

FIG. 1B is a disassembled view of the device.

FIG. 2A is a section view of the device.

FIG. 2B is an alternate section view of the device, with auxiliary shielding.

FIG. 3A is a plan view of a surface brachytherapy device with flexible attachment.

FIG. 3B is a plan view of the device in FIG. 3A, applied to a skin surface.

FIG. 3C is a plan view of the device in FIG. 3B, with the shield inserted into the base.

FIG. 4A is a plan view of a surface brachytherapy device, with alternate flexible attachment.

FIG. 4B is a section view of a surface brachytherapy device with flexible attachment.

FIG. 5 is a block diagram of a method for using a surface brachytherapy device.

FIG. 6 is a section view of a surface brachytherapy device, in an embodiment that uses magnetic coupling.

FIGS. 7A and 7B are section views of a surface brachytherapy device, in an embodiment that uses flexure coupling.

### DETAILED DESCRIPTION

The disclosure relates to an applicator device for surface brachytherapy. Surface brachytherapy is a radiotherapy technique used to treat a range of skin conditions including melanoma, lymphoma, basal cell and squamous cell (non-melanoma) sarcoma, Bowen's disease, actinic keratosis, keloids, and other malignant (cancerous) and non-malignant (precancerous or benign) disorders.

In high dose rate (HDR) surface brachytherapy, a source material (e.g., a beta-emitting radionuclide, or other suitable emitter) can be formed as a substantially planar or substantially curved radioactive patch or similar structure, and housed within an applicator designed to position the material adjacent the lesion or other skin area to be treated. The device can be adapted for accurate positioning and securement of a range of relatively smaller and relatively larger legions, or regular or irregular shape. The device can also provide shielding adapted to the emission characteristics of the selected radionuclide to be used for brachytherapy, in order to protect the surrounding tissues of the patient, and to protect caregivers from undesired exposure. Interstitial brachytherapy can be applied to deeper lesions, while more traditional “internal” brachytherapy techniques can be applied to other forms of cancer (e.g., prostate cancer), in either high dose rate (HDR) or low dose rate (LDR) applications.

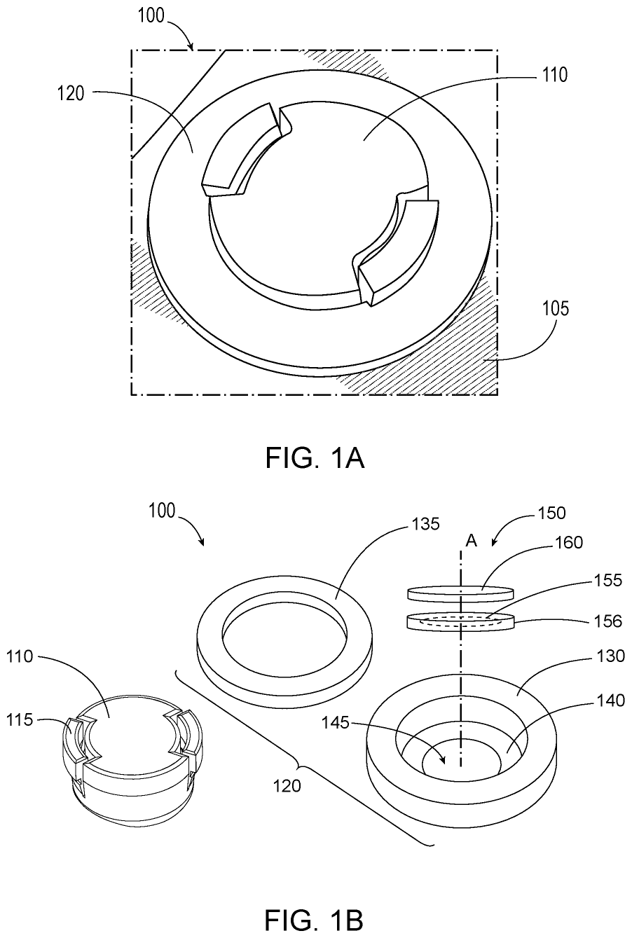

FIG. 1A top plan view of a surface brachytherapy applicator or device 100, as described herein. In this particular example, device 100 includes a shield body (or shield insert) 110 disposed in a shield guide or base 120, which is adapted to house a radioactive patch or other source. The base 120 can be coupled to a flexible fabric, band or similar attachment system 210, which is configured to secure the device 100 to a patient with a source accurately positioned inside the base 120, adjacent a skin lesion or other target area.

FIG. 1B is a disassembled view of device 100. As shown in FIG. 1B, base 120 is formed of a shield guide portion 130 and an adapter or interface portion 135, configured for attachment to the skin surface of a patient. A collimator 140 is disposed inside the adaptor/interface portion 135, coaxially oriented about axis A, and defining aperture 145.

Alternatively the collimator 140 may be absent, with the effective geometry of the aperture 145 defined by the physical extent of the source 155, or the source assembly 150 (or both). The radiotherapy patch (source assembly 150) can be provided with an encapsulated, beta-emitting, neutron-activated radionuclide or other radiation source 155, with level of activity, half-life, beta energy, gamma yield, and other emission characteristics selected for treating a particular skin lesion, or other condition.

The source material (or source) 155 can be encapsulated, for example by bonding a metal foil source 155 between layers of a table, impervious, radiation-resistant material 156. Source 155 can also be provided in a chemically and structurally stable, non-friable compound or matrix form, and sealed with an encapsulating material 156. An upper shield 160 can be glued, bonded, or otherwise attached to the top surface of the source 155, or provided as a separate component. Source assembly 150 may also include additional edge shielding, and other auxiliary shielding components can also be provided in shield body 110 and base 120, as described herein.

Shield guide 130 and adapter/interface 135 can be provided with a threaded coupling, friction fit, snap fitting, magnetic or mechanical attachment configured to engage the source assembly 150 adjacent collimator 140, with upper shield 160 disposed on the top surface (opposite the skin surface). Alternatively, shield guide 130 and adaptor/interface 135 can be joined with an adhesive or using magnetic coupling elements, or the guide 130 and adapter/interface 135 can be welded together or formed as a unit, providing a unitary, single-piece construction for base 120.

As shown in FIG. 1B, the shield body 110 is provided with one or more flexures (flexible tabs) or other flexible, biased engagements 115, adapted for secure engagement of the shield body 110 within the shield guide 130, and for securing the source assembly 150 into base 120. At this point, radiotherapy treatment may begin. Once treatment is complete (or at any other suitable time), the engagements 115 can be compressed to remove shield body 110, for access to source assembly 150.

In addition to the source 155 itself, source assembly 150 can also include one or more shield components, as described herein. Suitable examples for the construction of source 155 include, but are not limited to radioactive isotope skin patch devices as described in U.S. patent application Ser. No. 16/657,717, “Fabrication and Irradiation of a Radioactive Isotope Skin Patch,” filed Oct. 18, 2019 (U.S. Publication No. 2020/0188691 A1), and U.S. Provisional Patent Application No. 63/520,584, “Radiotherapy Devices including Beta Emitting Skin Patches and Methods for Quantifying Activity Thereof,” filed Aug. 18, 2023, each of which is incorporated by reference herein, in the entirety and for all purposes.

The source 155 can be activated in a radiation environment adapted to generate a radioisotope with selected half-life, gamma yield, and other radiation properties, for example by generating a beta emitting radioisotope via neutron capture. After activation, the source 155 can be provided with an upper shield 160, and/or edge shielding and other components to form the source assembly 150, and then inserted into the base 120 of device 100 for treatment by surface brachytherapy. Alternatively, one or more components of the device 100 can be adapted for exposure to the radiation environment with the source 155 in place, for example by selecting the materials of base 120, shield guide 130 and adapter/interface 135 for absorption cross section and radiation hardness, with or without shield body 110. The collimator 140, upper shield 160, and other higher-density components may also be assembled after activation of the source 155, in order to avoid the generation of other, undesired radioisotopes.

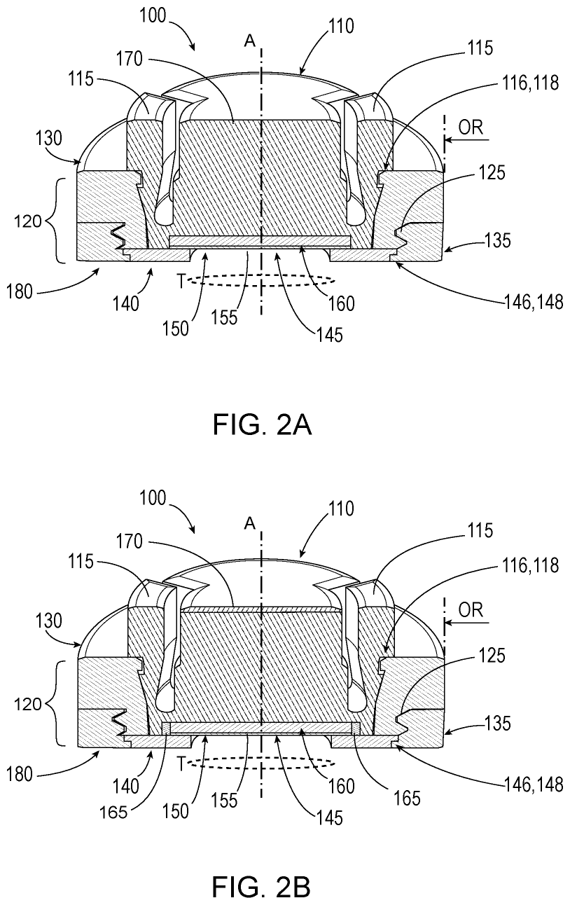

FIG. 2A is a section view of a surface brachytherapy applicator or device 100. As shown in FIG. 2A, collimator 140 is secured inside base 120 by a threaded coupling or other mechanical engagement 125 between the shield guide 130 and the adapter (or interface) 135. Depending on application, engagement 125 can also be formed as a friction fitting, mechanically biased fitting, using mechanical attachments such as screws or pins, or with magnetic coupling elements. The shield guide 130 and adapter/interface 135 can also be welded or glued together, or formed together as a unit, as described above.

For example, shield guide 130 can secure collimator 140 via a compressive axial loading onto a flange or similar coupling structure 146 defined along the outer circumference of collimator 140, and abutting a complementary ledge or land structure 148 defined along the inner circumference of adapter/interface 135. The position and shape of the coupling structures 146, 148 may also vary, and the complementary geometry may be reversed.

Once the shield guide 130, adapter/interface 135 and collimator are assembled, base 120 can be precisely located adjacent a skin lesion or other target area (T) on the skin surface, and attached using medical tape, adhesive, or other suitable material. Mechanical attachment features may also be used, for example hooks, snaps, buttons, or VELCRO-type hook and loop system, or magnetic coupling elements. An upper shield component 160 can be attached to the top surface of the source assembly 150, as described above, or coupled to the bottom surface of the main body portion of the shield body 110.

In any of the examples and embodiments described here, the upper shield 160 can also be embedded within the material of shield body 110, or provided as a separate component, or upper shield 160 may be absent. The source assembly 150, the upper shield 160, and the shield body 110 can typically be adhered or attached to each other in a single unit, or coupled together to form a substantially unitary structure. If the upper shield 160 is incorporated in the shield body 110, the source 155 can be adhered or attached directly to the shield body 110.

With the base 120 and collimator 140 secured into the desired location, the radioactive patch or other source assembly 150 can be inserted into the shield guide 130, flush with the collimator 140, defining an aperture 145 adjacent the target area (T). The source assembly 150 and source 155 may also at least partially define the aperture 145, together with or without a separate collimator 140. Radiation emitted by the source 155 is transmitted through aperture 145, and directed toward the target area (T). Other radiation, not directed through the aperture 145 toward the target area (T), is absorbed by the shield body 110, base 120, and collimator 140, (or any combination or sub-combination of these elements), reducing undesired exposure.

Plastic flexures or biased tab engagements 115 are disposed along the outer diameter or outer footprint of the shield body 110, allowing shield body 110 to snap into place by engagement within a complementary “snap” coupling features 116, 118 disposed on or along the outer surface of engagements 115 and the inner circumference of shield guide 130, as shown in FIG. 2A.

For example, a groove or channel feature 116 can be formed along the outer surfaces of engagements 115, and configured for a radially outward biasing engagement with a projecting tab or ridge structure 118 extending radially inward along the top circumference of shield guide 130. In some examples, biased engagements 115 and “snap” coupling features 116, 118 are adapted to attach shield body 110 to the shield guide 130 with an audible click or haptic signal, or both, indicating that the source assembly 150 is secured inside the base 120.

More generally, the complementary geometry of coupling features 116, 118 may be reversed, and the size, location and geometry may vary, for example using a threaded or frictional engagement, pins, tabs, mechanical attachments such as screws, or with magnetic coupling elements. The coupling features 116, 118 can also be adapted to key or clock the rotational position of the shield body 110 about axis A; e.g., in a preferred orientation with respect to shield guide 130, or in one or more selected orientations (e.g. at 180°, 90°, or other orientations). The engagement may also be unkeyed, for example by extending the tab or ridge features 118 continuously about the inner circumference on shield guide 130, so that the rotational position of shield body 110 is free to vary continuously about axis A with respect to base 120.

### Composition, Shielding and Contouring

The location, geometry and material composition of 100 may vary, depending on application. For example, a durable plastic, silicon, nylon, or other high-density polymer material can be used to form shield body 110 and base 120, including shield guide portion 130 and adapter/interface portion 135. The material can be selected for biocompatibility and suitability in medical applications, and with thickness and geometry selected to provide shielding from radiation emitted by source 155, which is not directed toward the skin lesion or other treatment surface.

Material properties such as melting point, hardness and flexibility can also be selected for repeated operation of the flexible engagements 115, when engaging and disengaging shield body 110 from shield guide 130, and for operation in a wide range of clinical, storage, sterilization, and activation environments. Higher-density metal, polymer and composite materials can also be used for the shield body 110 and base 120; e.g., aluminum or titanium, or a combination of structural and higher-density shielding materials, as described herein.

The collimator 140 and shield 160 can be formed of higher-density materials adapted for shielding from penetrating radiation emitted by the source 155; e.g., x rays (or x-rays) and gamma rays (or gamma-rays). Suitable shielding materials include stainless steel and other metals and metal alloys, selected based on material properties including machinability, stability, radiation hardness, and biocompatibility, as described above. Collimator 140 and shield 160 may also include other materials such as aluminum, titanium, magnesium, copper, iron, nickel, or chromium, or heavier materials such as tungsten, lead or barium compounds, for example in an encapsulated form, or in a stabilized composite matrix. In some examples and embodiments, the collimator 140 can be made from steel, including alloys such as 300-series stainless steels. In some examples and embodiments a magnetic ceramic or metal, such as a 400-series stainless steel alloy, may be preferred.

The adapter or interface 135 is configured to sit adjacent the patient's skin, and can be provided with a substantially flat or planar bottom surface (contact surface) 180, in a variety of sizes suitable for a range of clinical applications. Similarly, while device 100 is illustrated in a substantially round or cylindrical form that is merely a representative example. Device 100 can also be provided in oval, oblong, elongate, square, triangular, and other regular or irregular geometries, with one or more individual components configured accordingly (e.g., including but not limited to shield body 110, base 120, shield guide 130, adapter 135, collimator 140, aperture 145, source assembly 150, radiation source 155, an upper shield 160), for example in order to adapt device 100 for use on a particular patient, skin surface, and lesion or other target area geometry, or for other radiation treatment applications or fabrication processes.

Alternatively, the adapter/interface 135 can be provided with an arcuate, curved, or irregularly contoured contact surface 180, and configured to closely match the patient's skin surface, for example using a mold shaped to the skin surface, or by scanning the skin surface to generate a numerical model suitable for forming adapter/interface 135 by additive manufacturing. The outer radius (OR) of the shield guide 130 and adapter/interface 135 can also vary about the circumference of device 100, so that the outer footprint of the device 100 varies depending on the surrounding anatomical features (e.g., for treating legions on a patients face or head, near the ears, eyes, lips or nose, or other irregular or sensitive body features).

The source 155 and shield 160 can be formed as substantially flat structures adaptable to contact surfaces 180 of any shape, or curved to similarly match the curvature or contour of the contact surface 180 and/or the collimator 140, or the source 155 and shield 160 can be curved differently from the contact surface 180 or the collimator 140, or both. There can also be one or more gaps, spaces or air-gaps (G) between the source 155 and collimator 140, and the collimator 140 can have one or more such gaps (G) between the collimator 140 itself, and the target area (T) of the patient, which gap can vary in thickness.

In clinical applications, the devise is provided as a multicomponent system 100 including a base 120 configured for positioning a source assembly 150 and source 155 with respect to a target area (T), for example a skin lesion or other surface to be treated. The base includes an adapter or interface portion 135 with an aperture 145 defined by the collimator 140, and a shield guide 130 coupled to or defined on the adapter/interface portion 135, opposite the aperture 145.

For example, the collimator 140 can be secured between the adapter or interface portion 135 of the base 120, and the shield guide 130. Alternatively, collimator 140 may be absent, with aperture 145 defined directly in adapter/interface portion 135, or by the geometry of the shield body 110, shield guide 130, the source assembly 150 with source 155, or a combination thereof. In some examples and embodiments, the collimator 140 can be incorporated in or into the shield body 110, e.g., beneath the source 155, so that the collimator 140 is disposed between the source 155 and the target area (T).

The shield guide 130 is configured for coupling the shield body 110 with the base 120, and the adapter/interface portion 135 is configured for positioning the source assembly 150 and source 155 with respect to the target area (T). Shield body 110 is configured for coupling with the coupling (shield guide) portion 130, with the source assembly 150 and source 155 secured within the base 120, proximate or adjacent the aperture 145. The aperture 145 is configured to transmit radiation emitted by the source 155, where the radiation is directed toward target area (T). The base 120 and shield body 110 are configured to absorb at least a portion of other radiation emitted by the source 155, which is not directed toward the target area.

The collimator can be formed of or comprise a metal, metal alloy, or other material having a higher average density than that of the base 120, or a greater average atomic number (Z). The material can be selected for preferential absorption of penetrating radiation, including high-energy x rays and gamma rays. Suitable “high-Z” materials include metals, ceramics, and composites.

The aperture 145 may be substantially circular, rectangular, oval, or oblong, defining the geometry of the corresponding target area (T) according to the shape of the lesion to be treated. The aperture 145 may also have a substantially irregular geometry, adapted to transmit radiation emitted by the source 155 toward a target area with respectively irregular geometry.

The base 120 and shield body 110 are configured to reduce the flux of radiation (e.g., beta particles, x rays, gamma rays, etc.) that is not directed to the target area (T). The reduction can be by at least one order of magnitude, or at least two orders of magnitude, or three orders of magnitude or more, so that the exterior flux outside the device 100 approaches background. For example, the flux can be reduced along the top surface of the shield body 110, along the common axis A, and/or along the outer circumference of the base 120; e.g. in the plane of the source 155, extending about the axis A.

A number of flexures, tabs, or similar biased engagements 115 can be defined in or on the shield body 110, and configured for releasably coupling the shield body 110 with the base 120. For example, the engagement features maybe formed as flexures, tabs, prongs or resiliently biased structures, configured for releasably coupling the shield body 110 with the base 120. The engagement features can also be configured to generate an audio and/or haptic signal, which is responsive to the coupling (e.g., by snapping the shield body 110 and base 120 together). The signal thus indicates that the source is secured within the base 120.

### Additional Configurations

FIG. 2B is an alternate section view of device 100, with additional, auxiliary shielding 165, 170. Auxiliary shielding components 165, 170 can be formed of materials similar to collimator 140 and upper shield 160, as described above.

As shown in FIG. 2B, additional shielding 165 can be provided along the outer edges (outer circumference) of the source 155, in order to reduce the lateral flux of penetrating radiation, propagating transverse or perpendicular to axis A. For example, auxiliary edge shielding 165 can be provided as an annular body or ring disposed about the outer circumference of source 155 or upper shield 160, or both, and source 155, upper shield 160 and edge shielding 165 can be coupled together so that source assembly 150 can be inserted into *and removed from) base 120 as a unit.

Additional, auxiliary shielding 170 can also be disposed on the top surface of shield body 110, opposite the source assembly 150, in order to reduce the flux of penetrating radiation propagating along axis A. Auxiliary shielding 170 can also be embedded within shield body 110, either in combination with, or in place of, upper shield component 160, or provided in or on the flexures 115 and other components of the base 120, including the shield guide 130 and adapter/interface portion 135.

As shown in FIG. 2B, the additional shielding can thus include an upper shield 160 disposed on or adjacent the upper surface of the source 155, opposite the aperture 145, an auxiliary shield disposed on the top surface of (or at least partially embedded in) shield body 110, spaced from the source 155 along axis A, and an edge shield 165 disposed along the perimeter of the source 155, transverse to axis A. Similar to collimator 140, the upper shield 160, edge shield 165 and auxiliary shield 170 can be formed of or comprise a material with higher average density than that of the shield body 110 and base 120, or greater average atomic number (Z), selected for preferentially absorbing the penetrating radiation (e.g., x rays and gammas) emitted by the source 155.

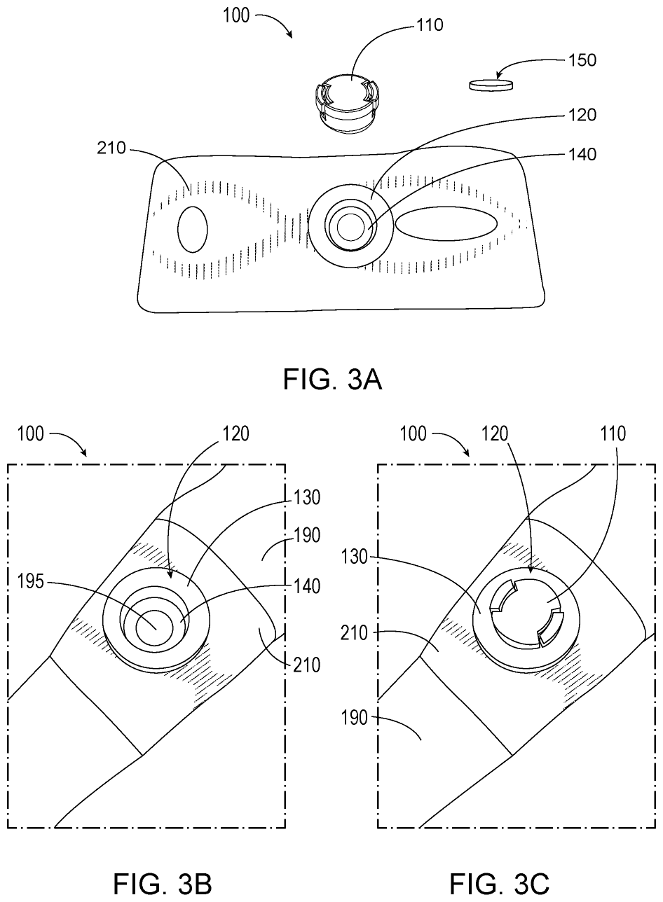

FIG. 3A is a plan view of a surface brachytherapy applicator or device 100, with the shield body 110 and source assembly 150 removed. A flexible attachment system 210 is coupled to base 120, for example in the form of flexible material such as medical, therapeutic or “sports” tape with a removable adhesive, or a flexible band with VELCRO type removable fasteners. Mechanical attachment features may also be used, for example hooks, snaps, buttons, or magnetic elements.

As shown in FIG. 3A, collimator 140 is disposed in base 120. Source assembly 150 includes a selected radioisotope or other source material, with or without an upper shield and auxiliary edge shielding components, as described above.

FIG. 3B is a plan view of device 100 according to FIG. 3A, applied to the patient's skin surface on an arm or other anatomical feature 190. FIG. 3C is a plan view of the device 100 according to FIG. 3A, with the shield body disposed in base 120, securing source assembly 150 into position for patient treatment.

The attachment system 210 can be configured for releasably securing the device 100 to one or more anatomical features 190 adjacent the target area, as defined below the base 120. Suitable attachment systems 210 include flexible fabric, flexible bands, flexible tapes, woven or non-woven fabrics (e.g., spun, blown, heat-sealed, wet-laid or dry-laid, punched, knitted or stitched), removable adhesives, magnetic elements, mechanical attachments, and any combination thereof.

As illustrated in FIGS. 3B and 3C, attachment system 210 can be used to secure brachytherapy device 100 to a patient, precisely positioning base 120 over a lesion or other target area adjacent the skin surface or anatomical features 190 to which the device 100 is attached. The source assembly 150 is then disposed inside the base 120, and secured by coupling the shield body 100 to the shield guide 130. Radiotherapy treatment can then take place to treat the lesion, while device 100 provides shielding to protect the surrounding tissues (and caregivers) from unwanted exposure.

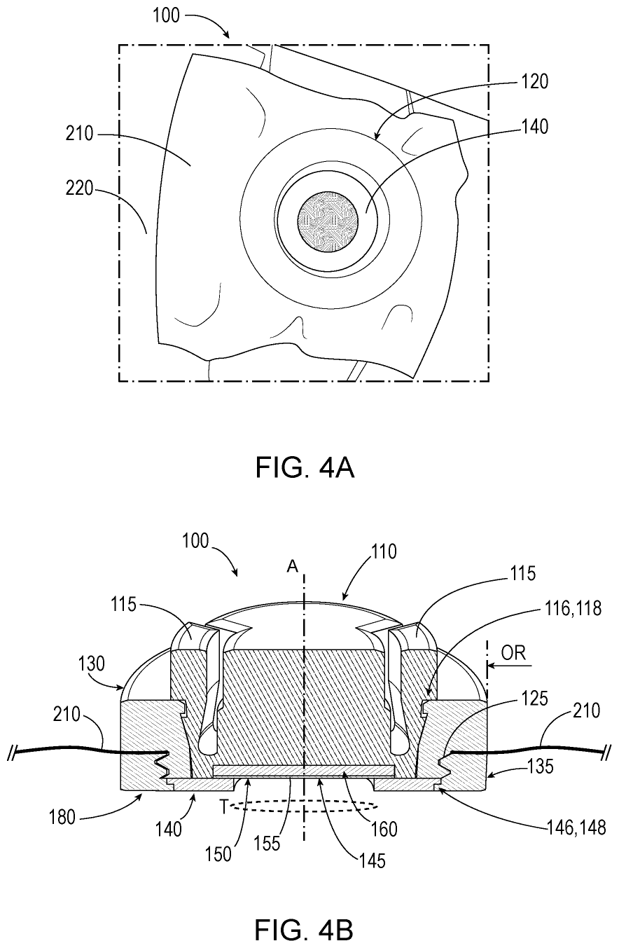

FIG. 4A is a plan view of a surface brachytherapy applicator or device 100, with alternate flexible attachment system 210 attached to base 120. In this configuration, attachment system 210 can be formed from a flexible, woven or nonwoven material (for example a breathable TYVEK type material, or another polymer or natural fiber material), and secured using medical tape 220, or a similar removable, biologically compatible adhesive material approved for clinical use. Mechanical attachment features may also be used, for example hooks or snap, a VELCRO-type hook and loop system, or magnetic attachments. Alternatively, attachment system 210 may have a substantially inflexible form, for example a substantially planar form, or attachment system 210 can be contoured for attachment to and treatment of a specific body surface; e.g., as described above for contouring the bottom surface of the base 120; e.g., along the bottom (contact) surface 180 of the adapter or interface portion 135.

FIG. 4B is a section view of device 100 with flexible attachment system 210. As shown in FIG. 4B, attachment system 210 can be “sandwiched” between the shield guide 130 and adapter/interface 135, and secured using a threaded coupling, a friction or snap fitting, magnetic coupling elements, or mechanical fasteners such as screws. The attachment system 210 can also be coupled to the bottom surface 180 of adapter/interface 135, or adapter/interface 135 and attachment system 210 can be formed together as a unit, for example in a contoured configuration adapted for positioning on a specific skin surface, as described above.

A contour can be defined along the bottom surface 180 of the adapter or interface portion 135 of the base 120, adjacent the collimator 140 and aperture 145, and adapted for coupling the base 120 to or along one or more anatomical features of the patient, adjacent the target area (T). Depending on the anatomy, the contour can be substantially planar, or a substantially spherical or substantially cylindrical section, or the contour can define one or more portions of a conic section.

For example, the contour can be adapted to one or more of a nose, lip, eye, brow or other facial feature, an ear, head or neck feature, a chest, torso or pelvic feature, a limb, hand or foot feature, a finger, toe or other digital feature, or any combination thereof. The outer footprint and thickness of the base 120 may also vary according to the adjacent anatomical geometry, as defined along the upper coupling (shield guide) portion 130, and the lower adapter/interface portion 135.

### Application Techniques

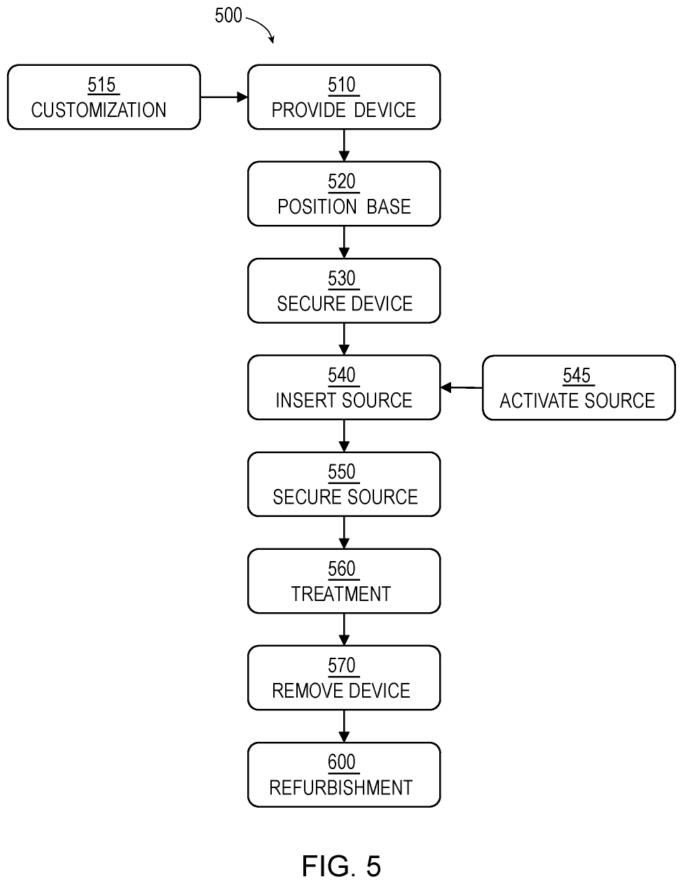

FIG. 5 is a block diagram of a method 500 for using a surface brachytherapy device, for example a system or device 100 for surface brachytherapy on a patient or other subject, as described herein. As shown in FIG. 5, method 500 includes one or more of providing (or acquiring) the device (step 510), positioning the base of the device with respect to a skin lesion or other surface (step 520), securing the device into position (step 530), inserting or adhering an activated radioisotope or other radiation source (or source assembly) into or onto the base or shield body (step 540), coupling the shield body with the base, in order to secure the source (step 550) for treatment (step 560), and removing the device from the subject (step 570).

Depending on application, method 500 may also include one or more of customizing the device (step 515), activating the source in a radiation environment (step 545), and removing the source assembly for reuse (step 600). Any or all of these steps can be repeated or performed in any order or combination, with or without additional techniques as described herein, and as known in the art.

In clinical applications, suitable brachytherapy devices can be provided (step 510) by a doctor, radiation specialist, or other caregiver. The device can be sized for a particular lesion, with a radiation source selected according to lesion type, size, depth, thickness, and other treatment considerations. A collimator and shielding components can be provided to direct the radiation toward the target area, absorbing other radiation to protect the patient and caregiver from unwanted exposure.

In many cases, the shield body with source inserted or adhered within would be stored and transported in a case where the shield body attaches to the case in a similar manner as to how it attaches to the base. This could be attached through engagement features comprising one or more flexures, tabs, prongs, magnets, or resiliently biased structures configured for releasably coupling the shield body with the base. The case would then shield the practitioner from any stray radiation during storage and transportation.

The base of the device is positioned (step 520) with respect to a skin lesion or other target area. The device can be provided in a cylindrical or oblong form, and positioned over a generally planar skin surface, or the base can be sized and contoured for application to a particular area of the patient's body; e.g., on the face, head, neck, or other sensitive area, which may have more complex geometry. This can be placed alone so that the area of body to be treated can be aligned with the aperture. In some cases, the aperture can be aligned visually with the target area, for example by a doctor or other medical practitioner.

The device can be secured (step 530) to a skin surface or other anatomical features using a removable adhesive, or with a flexible band or other attachment, or a combination thereof. Securing the device maintains precise positioning with respect to the lesion or other treatment surface, helping to reduce or minimize unwanted exposure to other tissues.

The source (or source assembly) is inserted into the base (step 540), so that radiotherapy can begin. The source can be provided in encapsulated form, or assembled together with additional upper, edge, or other shielding components, and inserted as a unit. In some applications, the shielding components can include the collimator.

The source assembly can be secured (step 550) by inserting the shield body into the base of the device. The shield body can be provided with flexible tabs or a similar biased coupling arrangement, which engages the shield body with an audible click or visual or haptic signal (or combination thereof) to indicate that the shield body is locked into or onto the shield guide, with the source assembly secured inside the base for radiotherapy treatment (step 560).

Radiation emitted by the source is directed toward the target area through the aperture, for treatment of the lesion (step 560). Radiation that is not directed to the target area is absorbed by the base and shield body, reducing unwanted exposure. The device can be removed (step 570) following treatment, or at any other suitable time.

In some applications, the device can be customized (step 515) according to a particular patient's condition. Customization can include selecting the source activity, geometry and emission characteristics based on the type and size of lesion to be treated, selecting a collimator to define the aperture according to the lesion geometry, and contouring the contact surface to conform to the adjacent anatomical features. The base width and radius can also be varied to accommodate the adjacent anatomy, and additional (e.g., higher-density) materials can be provided to reduce penetrating radiation exposure, further shielding the adjacent body features from undesired exposure.

In some applications, the source can be activated (step 545) in or near the clinical setting, for example to reduce storage time and allow for a wider range of radioisotopes to be used. For example, this approach can be used to accommodate radioisotopes with shorter half-lives, or when customization of the device may be better accomplished by activating the source after it has been disposed in the base.

After removing the device (step 570), the source assembly can be refurbished (step 600) for reuse, according to any of the examples and embodiments described here.

Refurbishment can be performed after a number of half-lives of the radioisotope have elapsed, reducing emissions toward the background level. The shielding materials can be removed, and the source material can then be activated again (step 545), for use with the same (or other) device (returning to step 510). This approach contrasts with other, single-use source techniques, reducing hazardous waste and associated disposal costs.

In all of these applications, the device 100 described here provides a number of additional advantages over the prior art, including the enablement of beta-emitting isotope sources 155 for high dose rate (HDR) superficial (surface) brachytherapy, as opposed to traditional sealed source techniques. The applicator base 120 can be positioned without the radiation source (activated radioisotope) 155 present, in order to more accurately define the radiation field with respect to the skin lesion or other treatment surface adjacent the anatomical feature 190 to which the device is attached. The source assembly 150 and source 155 need not be inserted into the base 120 until all other procedures for commencing therapy are complete, reducing undesired exposure for both the patient and caregivers.

The biased “snapping” flexures (flexible tab) attachments 115 coupling the shield body 110 to the base 120 make securing the source assembly 150 and source 155 quick and easy, which also reduces unwanted exposure to the patient and staff. With the shield body 110 in place, the thickness of the shield guide 130, adapter/interface 135 and other base components of base 120 can be adapted to stop substantially all beta radiation from the source, which is not intentionally directed toward the skin lesion or other target area.

Higher density (e.g., metal) collimator 140 and shield components 160, 165, 170 can also be provided to help reduce unwanted penetrating radiation (e.g., beta particles, x rays and gammas), which may all be emitted by the radioactive source 155. The device 100 can also be made from low-cost, biocompatible plastics and biocompatible metals such as stainless steel (e.g., 316 stainless steel). The devices 100 illustrated in the figures can also be molded or additively manufactured from biocompatible plastics, resins and other matrix materials; e.g., using a commercially available 3D printing system.

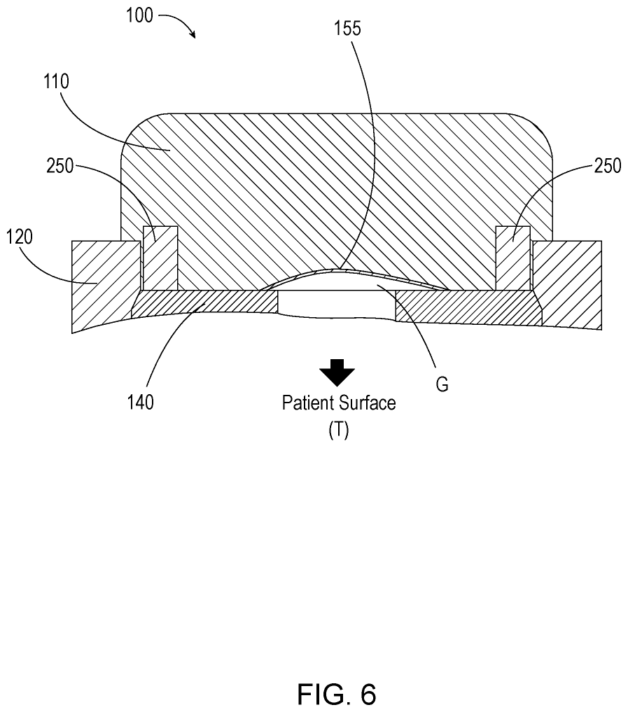

FIG. 6 is a section view of a surface brachytherapy device 100, in an embodiment that uses magnetic coupling elements 250. In this particular example, device 100 does not include a high-Z shielding layer (which could be incorporated into the shield body 110, either alternatively or in combination with the other features that are shown). Device 100 has a number of curved or contoured elements, including but not limited to the base 120, collimator 140, shield body 110 or upper shield 160 (or both), and the brachytherapy source assembly 160 and source 155. For example, one or more elements 110, 120, 140, 150, 155, 160 can be provided with non-planar, curved surfaces, adapted to the corresponding contour of the patient's anatomy at the treatment site.

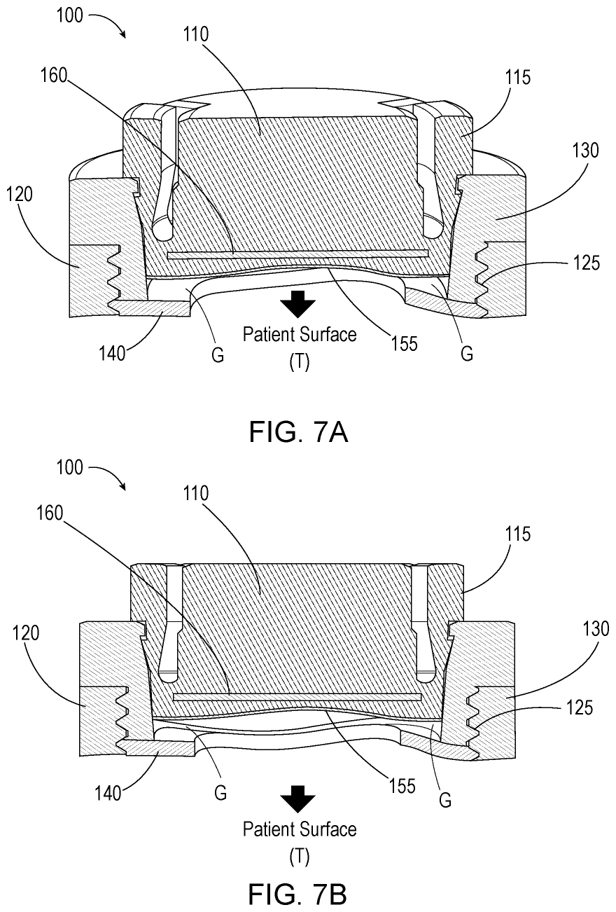

FIGS. 7A and 7B are section views of a surface brachytherapy device 100, in an embodiment that uses flexure couplings and has curved elements, including, but not limited to, for the base 120, the collimator 140, the shield 160 and the brachytherapy source 155, and a space, air-gap or other gap (G) between the source 155 and the collimator 140. The curved elements can be adapted to conform to the contour of the patient's anatomy at the treatment site, as described above.

### EXAMPLES

Suitable systems as described here can comprise a base configured for positioning a radiation source with respect to a target area, the base having an aperture defined therein, and a shield body configured for coupling with the base, where the source is securable proximate or adjacent the aperture. The aperture can be configured to transmit radiation emitted by the source, for example where said radiation is directed toward the target area for surface brachytherapy or other radiation treatment. The base and shield body can be configured to absorb at least a portion of other radiation emitted by the source, which is not directed toward the target area.

In any of these examples and embodiments, the base can comprise an adapter or interface portion having the aperture defined therein, and a shield guide portion coupled to or defined on the adapter or interface portion, opposite the aperture. The adapter or interface portion can be configured for positioning the source with respect to the target area, and the shield guide portion can be configured for coupling the shield body with the base.

In any of these examples and embodiments, the source can be securable within the shield body and the shield body can be configured for securing the source with the base, or the source can be adhered to or within the shield body. Independently or in combination with these configurations, the source can be disposed proximate or adjacent the aperture.

In any of these examples and embodiments can further comprise a collimator disposed between the source and the target area, for example where the collimator defines the aperture, or where the collimator is coupled to the base or the shield body, or where the collimator is secured between the adapter or interface portion of the base and the shield guide portion of the base, of any combination thereof.

In any of these examples and embodiments, the collimator can be formed of or comprise a material having a higher average density or greater average atomic number (Z) than that of the base.

In any of these examples and embodiments, the aperture can be substantially circular, rectangular, oval, or oblong, or the aperture can be at least partially defined according to a geometry of the source, or the aperture can have a substantially irregular geometry adapted to transmit said radiation emitted by said source directed toward a respectively geometry of the target area, or any combination.

In any of these examples and embodiments, one or more of the base, collimator, and/or adapter can comprise or be made of a 300-series stainless steel, a 400-series stainless steel, or a composite made of a polymer or volume printed or additively manufactured material impregnated with tungsten, lead, or other high-Z particulates, or a combination thereof.

In any of these examples and embodiments, one or more of the base, the adapter, the collimator, and/or the source can have a substantially non-planar or curved surface, or the device can further comprise one or more gaps disposed between the source and the target area, and/or the source and the adapter or collimator, or any combination of said substantially non-planar or curved surface(s) and/or gap(s) can be included.

In any of these examples and embodiments, the base and shield body can be configured to reduce a flux of radiation that is not directed to the target area by at least one order of magnitude, or at least two orders of magnitude. For example, the radiation can comprise x ray, gamma ray, alpha particle or beta particle radiation, or a combination thereof. For example, the flux of radiation can be reduced by at least said order or orders of magnitude at a top surface of the shield body, along a common axis of the base and shield body, and/or along an outer circumference of the base, extending about said common axis.

In any of these examples and embodiments, the base and aperture can define a common transverse axis and the device can further comprise one or more of an upper shield disposed on or adjacent an upper surface of the source, opposite the aperture along the axis, an edge shield disposed about or along a perimeter of the source, extending transverse to the axis, or an auxiliary shield disposed on or at least partially embedded in the shield body, spaced from the source along the axis, or any combination thereof.

In any of these examples and embodiments, one or more of the upper shield, the edge shield and the auxiliary shield can be formed of or comprise a material having a higher average density or greater average atomic number (Z) than that of the base. For example, the material can be selected for preferentially absorbing a penetrating component of the radiation emitted by the source, as compared to absorption of the penetrating component by the base; e.g., where the penetrating component comprising x rays or gamma rays, or penetrating beta particles, or any combination thereof.

In any of these examples and embodiments, the device can further comprise one or more engagement features or engagement elements disposed or defined in or on the shield body. For example, the engagement features or engagement elements can include mechanical, resiliently biased or magnetic elements, and they can be configured for releasably coupling the shield body with the base.

In any of these examples and embodiments, the engagement features or engagement elements can comprise one or more magnets, flexures, tabs, prongs, or resiliently biased structures configured for releasably coupling the shield body with the base, or one or more threaded couplings, magnetic coupling elements, or friction or snap fittings, or any combination thereof. The , wherein the engagement features are configured to generate an audio, visual, and/or haptic signal responsive to releasably coupling the shield body with the base, wherein the signal is indicative of securing the source within the base.

In any of these examples and embodiments, the engagement features or engagement elements can be configured to generate an audio, visual, and/or haptic signal responsive to releasably coupling the shield body with the base, for example where the signal is indicative of securing the source within the base.

Any of these examples and embodiments can include or comprise an attachment system coupled to the base, for example where the attachment system is configured for releasably securing the device to one or more anatomical features adjacent the target area. The attachment system can comprise one or more of a flexible fabric, a flexible band, a flexible tape, a woven or non-woven fabric, magnetic coupling elements, mechanical fasteners, a VELCRO-type hook and loop fastening system, an adhesive material, or a removable adhesive material.

In any of these examples and embodiments, a contour can be defined along a bottom surface of the base, collimator, and/or adapter adjacent the aperture, for example where the contour is adapted for coupling the base, collimator, and/or adapter to or along one or more anatomical features defined adjacent the target area. The contour can be substantially planar or include a substantially spherical or substantially cylindrical section, or the contour can define one or more portions of a conic section.

In any of these examples and embodiments, the contour of the device can be adapted to one or more anatomical features, for example as selected from a nose, lip, eye, brow or other facial feature, an ear, head or neck feature, a chest, torso or pelvic feature, a limb, hand or foot feature, a finger, toe or other digital feature, or any combination thereof. An outer footprint, thickness or geometry of one or more of the base, collimator, shield, adapter and source can vary according to the anatomical geometry.

Suitable method according to this disclosure can comprise positioning a device with respect to a target area on a subject, the device having a base with an aperture defined therein, disposing the device adjacent one or more anatomical features of the subject, inserting a source into the base, wherein radiation emitted by the source is directed toward the target area through the aperture, and coupling a shield body to the base. The source can be disposed in or adjacent the base, or secured or adhered within the base, and at least a portion of radiation emitted by the source that is not directed to the target area can be absorbed by the base and shield body.

These method steps can be performed in any order or combination, with or without additional processes. For example, the method can further comprise activating the source in a radiation environment, where the radiation emitted by the source comprises x rays, gamma rays, alpha particles, beta particles, or a combination thereof.

Any of these methods can further comprise providing a collimator in the base, for example where the source is disposed adjacent the collimator, where the collimator defines the aperture, and where the aperture defines a geometry of the target area, or visually aligning the aperture defined by the collimator or base to the target area, before the source is inserted.

Any of these methods can further comprise providing a contour on the bottom surface of the base, collimator, and/or adapter, for example adjacent the aperture, where the contour is adapted to secure the device to the one or more anatomical features of the subject.

In any of these examples and embodiments, the source can be attached to the shield and disposed adjacent the base, or the method can further comprise one or more of removing the device from the subject, decoupling the shield body from the base, and/or removing the source from the base.

This disclosure is made with reference to representative examples and embodiments. Changes can be made and equivalents can be substituted to adapt these teachings to other materials, problems and applications, as known to persons of skill in that art, while remaining within the scope of invention as defined by the appended claims.

1. A system comprising:
a base configured for positioning a radiation source with respect to a target area, the base having an aperture defined therein; and
a shield body configured for coupling with the base, wherein the source is securable proximate or adjacent the aperture;
wherein the aperture is configured to transmit radiation emitted by the source, wherein said radiation is directed toward the target area; and
wherein the base and shield body are configured to absorb at least a portion of other radiation emitted by the source, which is not directed toward the target area.

2. The system of claim 1, wherein the base comprises an adapter or interface portion having the aperture defined therein and a shield guide portion coupled to or defined on the adapter or interface portion, opposite the aperture, wherein the adapter or interface portion is configured for positioning the source with respect to the target area and the shield guide portion is configured for coupling the shield body with the base.

3. The system of claim 2, wherein the source is securable within the shield body and the shield body is configured for securing the source with the base, or wherein the source is adhered to or within the shield body, such that the source is disposed proximate or adjacent the aperture.

4. The system of claim 2, further comprising a collimator disposed between the source and the target area;
wherein the collimator defines the aperture; and
wherein the collimator is coupled to the base or the shield body, or
wherein the collimator is secured between the adapter or interface portion of the base and the shield guide portion of the base.

5. The system of claim 4, wherein the collimator is formed of or comprises a material having a higher average density or greater average atomic number (Z) than that of the base.

6. The system of claim 1, wherein:
the aperture is substantially circular, rectangular, oval, or oblong; or
the aperture is at least partially defined by a geometry of the source, or with a substantially irregular geometry adapted to transmit said radiation emitted by said source directed toward a respectively geometry of the target area, or both.

7. The system of claim 4, wherein one or more of the base, collimator, and/or adapter comprises or is made of a 300-series stainless steel, a 400-series stainless steel, or a composite made of a polymer or volume printed or additively manufactured material impregnated with tungsten, lead, or other high-Z particulates, or a combination thereof.

8. The system of claim 4, wherein:
one or more of the base, the adapter, the collimator, and/or the source has a substantially non-planar or curved surface; or
further comprising one or more gaps between the source and the target area, and/or the source and the adapter or collimator; or
any combination thereof.

9. The system of claim 1, wherein the base and shield body are configured to reduce a flux of radiation that is not directed to the target area by at least one order of magnitude, or at least two orders of magnitude, and:
wherein the radiation comprises x ray, gamma ray, alpha particle or beta particle radiation, or a combination thereof; or
wherein the flux of radiation is reduced by at least said order or orders of magnitude at a top surface of the shield body, along a common axis of the base and shield body, and/or along an outer circumference of the base, extending about said common axis.

10. The system of claim 1, wherein the base and aperture define a common transverse axis and further comprising one or more of:
an upper shield disposed on or adjacent an upper surface of the source, opposite the aperture along the axis;
an edge shield disposed about or along a perimeter of the source, extending transverse to the axis;
an auxiliary shield disposed on or at least partially embedded in the shield body, spaced from the source along the axis.

11. The system of claim 10, wherein one or more of the upper shield, the edge shield and the auxiliary shield is formed of or comprises a material having a higher average density or greater average atomic number (Z) than that of the base, and wherein the material is selected for preferentially absorbing a penetrating component of the radiation emitted by the source, as compared to absorption of the penetrating component by the base, wherein the penetrating component comprising beta particles, x rays, gamma rays, or a combination thereof.

12. The system of claim 1, further comprising one or more engagement features or engagement elements disposed or defined in or on the shield body, wherein the engagement features or engagement elements are configured for releasably coupling the shield body with the base.

13. The system of claim 12, wherein the engagement features or engagement elements comprise:
one or more magnets, flexures, tabs, prongs, or resiliently biased structures configured for releasably coupling the shield body with the base; or
one or more threaded couplings, magnetic coupling elements, or friction or snap fittings; or
any combination thereof.

14. The system of claim 12, wherein the engagement features or engagement elements are configured to generate an audio, visual, and/or haptic signal responsive to releasably coupling the shield body with the base, wherein the signal is indicative of securing the source within the base.

15. The system of claim 1, further comprising an attachment system coupled to the base, wherein the attachment system is configured for releasably securing the device to one or more anatomical features adjacent the target area.

16. The system of claim 15, wherein the attachment system comprises one or more of a flexible fabric, a flexible band, a flexible tape, a woven or non-woven fabric, magnetic coupling elements, mechanical fasteners, a VELCRO-type hook and loop fastening system, an adhesive material, or a removable adhesive material.

17. The system of claim 1, further comprising a contour defined along a bottom surface of the base, collimator, and/or adapter adjacent the aperture, wherein the contour is adapted for coupling the base, collimator, and/or adapter to or along one or more anatomical features defined adjacent the target area.

18. The system of claim 17, wherein the contour is substantially planar or comprises a substantially spherical or substantially cylindrical section, or wherein the contour defines one or more portions of a conic section.

19. The system of claim 17, wherein the contour is adapted to one or more anatomical features selected from a nose, lip, eye, brow or other facial feature, an ear, head or neck feature, a chest, torso or pelvic feature, a limb, hand or foot feature, a finger, toe or other digital feature, or a combination thereof.

20. The system of claim 19, wherein an outer footprint, thickness or geometry of one or more of the base, collimator, shield, adapter and source varies according to the anatomical geometry.

21. A method comprising:
positioning a device with respect to a target area on a subject, the device having a base with an aperture defined therein;
disposing the device adjacent one or more anatomical features of the subject;
inserting a source into the base, wherein radiation emitted by the source is directed toward the target area through the aperture;
coupling a shield body to the base, wherein the source is disposed in or adjacent the base, or secured or adhered within the base, and at least a portion of radiation emitted by the source that is not directed to the target area is absorbed by the base and shield body.

22. The method of claim 21, further comprising activating the source in a radiation environment, wherein the radiation emitted by the source comprises x rays, gamma rays, alpha particles, beta particles, or a combination thereof.

23. The method of claim 21, further comprising:
providing a collimator in the base, wherein the source is disposed adjacent the collimator, wherein the collimator defines the aperture, and wherein the aperture defines a geometry of the target area; or
visually aligning the aperture defined by the collimator or base to the target area before the source is inserted.

24. The method of claim 21, further comprising providing a contour on the bottom surface of the base, collimator, and/or adapter, adjacent the aperture, wherein the contour is adapted to secure the device to the one or more anatomical features of the subject.

25. The method of claim 21, wherein the source is attached to the shield and disposed adjacent the base, or further comprising one or more of removing the device from the subject, decoupling the shield body from the base, and removing the source from the base.
